(19) United States
(12) Patent Application Publication
(10) Pub. No.: US 2010/0183638 A1
(43) Pub. Date: Jul. 22, 2010

(54) RESTRICTIVE AGONIST OF TOLL-LIKE RECEPTOR 3 (TLR3)

(76) Inventor: Brian B. Gowen, Millville, UT (US)

Correspondence Address: NIXON & VANDERHYE, PC, 901 NORTH GLEBE ROAD, 11TH FLOOR, ARLINGTON, VA, 22203, US

(21) Appl. No.: 12/530,148

(22) PCT Filed: Mar. 5, 2008
(86) PCT No.: PCT/US08/02874

§ 371 Date: Apr. 5, 2010

Related U.S. Application Data
(60) Provisional application No. 60/904,792, filed on Mar. 5, 2007.

(51) Int. Cl. A61K 31/713; A61K 39/39; A61P 31/14

(52) U.S. Cl. 424/184.1; 424/278.1

(57) ABSTRACT

A mismatched double-stranded ribonucleic acid, which is an agonist for Toll-like receptor 3 (TLR3), is used in vitro or in vivo as one or more of antiviral agent, antiproliferative agent, and immunostimulant. Methods of medical treat-ment and processes for manufacturing medicaments are provided.

### CROSS-REFERENCE TO RELATED APPLICATION

This application claims the priority benefit of provisional U.S. Application No. 60/904,792, filed Mar. 5, 2007.

### FEDERALLY-SPONSORED RESEARCH OR DEVELOPMENT

The U.S. government has certain rights in this invention as provided for by the terms of NIH-NO1-AI-15435, which was awarded by the Department of Health and Human Services.

### FIELD OF THE INVENTION

This invention relates to providing an agonist for Toll-like receptor 3 (TLR3) for use as antiviral agent, antiproliferative agent, immunostimulant, or any of their combinations. Methods of medical treatment and processes for manufacturing medicaments are provided.

### BACKGROUND OF THE INVENTION

Double-stranded RNA like poly(I:C) has been used as a TLR3 agonist. But its usefulness as a medicament is limited by its toxicity. Improved medicaments are thus sought that can be used as an antiviral agent, antiproliferative agent, and/or immunostimulant by specifically targeting TLR3, instead of other receptors belonging to this family. For example, a desirable medicament would have an increased therapeutic index (e.g., the ratio of the dose that produces a toxic effect divided by the dose that produces a therapeutic effect such as LD₅₀ divided by ED₅₀) for treating an incipient or established infection, treating a precancerous or cancerous condition, or inducing an inflammatory response as mediated by TLR3.

Double-stranded ribonucleic acid (dsRNA) triggers innate immunity (e.g., the production of interferon and other cytokines) through dsRNA-dependent intracellular antiviral defense mechanisms including the 2′,5′-oligoadenylate synthetase/RNase L and p68 protein kinase pathways. AMPLIGEN® poly(I:C₁₂U) from HEMISPHERx® Biopharma is a specifically-configured dsRNA with antiviral and immunostimulatory properties, but which exhibits reduced toxicity. AMPLIGEN® poly(I:C₁₂U) inhibits viral and cancer cell growth through pleiotropic activities: it regulates 2′,5′-oligoadenylate synthetase/RNase L and p68 protein kinase pathways as do other dsRNA molecules. Here, poly(I:C₁₂U) is discovered to mediate its effects in the body by acting as a specific agonist of TLR3.

Therefore, it is an objective to provide treatment for a patient in need of an antiviral agent, antiproliferative agent, and/or immunostimulant. A long-felt need for a selective TLR3 agonist is addressed thereby. Methods for treating subjects and processes for making medicaments, especially involving infectious disease, cell proliferation, and/or vaccination, are provided. Further objectives and advantages of the invention are described below or would be apparent to a person skilled in the art from that discussion.

### SUMMARY OF THE INVENTION

The invention may be used to treat a subject (e.g., human or animal) with an incipient or established viral infection, a pathological condition marked by abnormal cell proliferation (e.g., neoplasm or tumor), or as an immunostimulant to vaccinate the subject against viral infection. It is preferred that the amount of mismatched double-stranded ribonucleic acid (dsRNA) used is sufficient to bind Toll-Like Receptor 3 (TLR3) on immune cells of the subject. Innate immunity may be triggered thereby. In particular, a specifically-configured dsRNA may be used to activate TLR3 without activating other Toll-like receptors like TLR4 or an RNA helicase like RIG-I or mda-5.

The subject may be infected with a virus, especially a bunyavirus or more particularly a phlebovirus. A pharmaceutical composition which is comprised of specifically-configured dsRNA in an amount sufficient to bind to TLR3 is administered to the subject. Viral infection of the subject is reduced or eliminated thereby as assayed by decreased recovery time, increased immunity (e.g., increase in antibody titer, lymphocyte proliferation, killing of infected cells, or natural killer (NK) cell activity), decreased virus number of replication, or a combination thereof as compared to the subject not treated with specifically-configured dsRNA.

The subject may be afflicted by abnormal cell proliferation (e.g., neoplasm or tumor, other transformed cell). It may be that a pharmaceutical composition, which is comprised of specifically-configured dsRNA in an amount sufficient to bind to TLR3, is administered to the subject. Cell proliferation is reduced, neoplastic cells are eliminated, and/or morbidity or mortality of the subject is improved thereby as compared to the condition of a subject not treated with specifically-configured dsRNA.

The subject may be vaccinated against the virus or neoplasm. Immediately before, during, or immediately after vaccination, a pharmaceutical composition which is comprised of specifically-configured dsRNA in an amount sufficient to bind to TLR3 is administered to the subject. The immune response to the vaccine is stimulated thereby. The vaccine may be comprised of a killed or attenuated virus, fraction of a neoplastic cell, one or more isolated viral proteins, or one or more isolated tumor antigens. An in situ vaccine may be comprised of antigen produced at the site and the specifically-configured dsRNA acting as an adjuvant thereon. The virus may be a bunyavirus, more particularly a phlebovirus.

Antigen presenting cells (e.g., dendritic cells, macrophages) and mucosal tissues (e.g., gastric or respiratory epithelium) are preferred targets in the body for the specifically-configured dsRNA. The virus or tumor may be presented, and the antigen should be susceptible to the sole action of the specifically-configured dsRNA acting exclusively as a TLR3 agonist. The specifically-configured dsRNA is preferably administered by intravenous infusion; intradermal, subcutaneous, or intramuscular injection; intranasal or intratracheal inhalation; or oropharyngeal exposure.

Also provided are processes for using and making medicaments. It should be noted, however, that a claim directed to the product is not necessarily limited to these processes unless the particular steps of the process are recited in the product claim.

Further aspects of the invention will be apparent to a person skilled in the art from the detailed description and claims, and generalizations thereto.

### BRIEF DESCRIPTION OF THE DRAWINGS

FIG. 1 shows that poly(I:C₁₂U) treatment limits liver disease and systemic virus burden in wild-type but not TLR3^{-/-} mice. Groups of 8 week-old TLR3^{-/-} (FIGS. 1A, 1C, 1G and 1E) and wild-type (FIGS. 1B, 1D, 1E and 1F) mice were virally challenged with PTV (day 0) and treated with 10 μg of poly(I:C₁₂U) or saline placebo 24 hours after infection. Shown are mean serum ALT levels (FIGS. 1A-1B), liver scores (FIGS. 1C-1D) liver virus titers (FIGS. 1E-1F) and serum virus titers (FIGS. 1G-1H) for samples collected on the indicated days post-infection. Data represent the mean and standard deviation of five animals per group. poly(I:C₁₂U), mdsRNA. IU, international units. *P<0.05; **P<0.01 compared to saline-treated controls.

FIG. 2 shows the induction of IFN-β in uninfected TLR3^{-/-} and wild-type mice following poly(I:C₁₂U) exposure. Groups of 8 week-old mice were injected i.p. with 10 μg of poly(I:C₁₂U) and systemic IFN-β levels were determined for serum samples collected at the indicated times post-exposure. Data represent the mean and standard deviation of three animals per group.

### DESCRIPTION OF SPECIFIC EMBODIMENTS OF THE INVENTION

The invention may treat an infection by an RNA virus belonging to Group III, Group IV, or Group V of the Baltimore classification system. It possesses ribonucleic acid (RNA) as its genetic material and does not replicate using a DNA intermediate. The RNA is usually single stranded (ssRNA) but can occasionally be double stranded (dsRNA). RNA viruses can be further classified according to the sense or polarity of their RNA into negative-sense and positive-sense RNA viruses. Positive-sense viral RNA is identical to viral mRNA and thus can be immediately translated by the host cell. Negative-sense viral RNA is complementary to mRNA and thus must be converted to positive-sense RNA by an RNA polymerase before translation. As such, purified RNA of a positive-sense virus can directly cause infection though it may be less infectious than the whole virus particle. Purified RNA of a negative-sense virus is not infectious by itself as it needs to be transcribed into positive-sense RNA.

RNA viruses that infect humans and animals include those belonging to the Birnaviridae and Reoviridae families (Group III dsRNA viruses); the Arteriviridae, Astroviridae, Caliciviridae, Hepevindae, and Roniviridae families (Group IV positive-sense ssRNA viruses); and the Arenaviridae, Bornaviridae, Bunyaviridae, Filoviridae, Paramyxoviridae, and Rhabdoviridae families (Group V negative-sense ssRNA viruses). Specifically-configured, double-stranded ribonucleic acid (dsRNA) is also known to treat infection by RNA viruses from the Flaviviridae, Hepadnaviridae, Orthomyxoviridae, Picornaviridae, Retroviridae, and Togaviridae families. Viruses of these families may or may not be included within the scope of the invention.

Cells of the subject undergoing abnormal proliferation may be a neoplasm or tumor (e.g., carcinoma, sarcoma, leukemia, lymphoma), especially cells transformed by a tumor virus (e.g., DNA or RNA virus carrying a transforming gene or oncogene) or otherwise infected by a virus associated with cancer. For example, Epstein-Barr virus is associated with nasopharyngeal cancer, Hodgkin's lymphoma, Burkitt's lymphoma, and other B lymphomas; human hepatitis B and C viruses (HBV and HCV) are associated with liver cancer; human herpesvirus 8 (HHV8) is associated with Kaposi's sarcoma; human papillomaviruses (e.g., HPV6, HPV11, HPV16, HPV18, or combination thereof) are associated with cervical cancer, anal cancer, and genital warts; and human T-lymphotrophic virus (HTLV) is associated with T-cell leukemia and lymphoma. Cancers include those originating from the gastrointestinal (e.g., esophagus, colon, intestine, ileum, rectum, anus, liver, pancreas, stomach), genitourinary (e.g., bladder, kidney, prostate), musculoskeletal, nervous, pulmonary (e.g., lung), or reproductive (e.g., cervix, ovary, testicle) organ systems.

Poly(riboinosinic) is partially hybridized to poly(ribocytosinic₁₂uracilic) and can be represented as rIₙ•r(C₁₂U)ₙ. Other specifically-configured dsRNA that may be used are based on copolynucleotides selected from poly(CₙU) and poly(CₙG) in which n is an integer from 4 to 29 or are mismatched analogs of complexes of polyriboinosinic and polyribocytidilic acids, formed by modifying rIₙ•rCₙ to incorporate unpaired bases (uracil or guanine) along the polyribocytidylate (rCₙ) strand. Alternatively mismatched dsRNA may be derived from r(I)•r(C) dsRNA by modifying the ribosyl backbone of polyriboinosinic acid (rIₙ), e.g., by including 2′-O-methyl ribosyl residues. Mismatched dsRNA may be complexed with an RNA-stabilizing polymer such as lysine cellulose. Of these mismatched analogs of rIₙ•rCₙ, the preferred ones are of the general formula rIₙ•r(C₁₁₋₁₄U)ₙ and are described in U.S. Pat. Nos. 4,024,222 and 4,130,641; which are incorporated by reference. The dsRNA described therein generally are suitable for use according to the present invention. See also U.S. Pat. No. 5,258,369.

Specifically-configured dsRNA may be administered by any suitable local or systemic route including enteral (e.g., oral, feeding tube, enema), topical (e.g., patch acting epicutaneously, suppository acting in the rectum or vagina), and parenteral (e.g., transdermal patch; subcutaneous, intravenous, intramuscular, intradermal, or intraperitoneal injection; buccal, sublingual, or transmucosal; inhalation or instillation intranasally or intratracheally). The nucleic acid may be micronized for inhalation, dissolved in a vehicle (e.g., sterile buffered saline or water) for injection or instillation, or encapsulated in a liposome or other carrier for targeted delivery. Preferred are carriers that target the nucleic acid to the TLR3 receptor on antigen presenting cells and epithelium. A medicament may be formulated as a pharmaceutical composition containing at least an effective amount of specifically-configured dsRNA manufactured (and optionally stored) under aseptic conditions and tested for low microbial and endotoxin contamination. The medicament may further contain a physiologically-acceptable vehicle or carrier. It will be appreciated that the preferred route may vary with condition and age of the subject, the nature of the infectious or neoplastic disease, and the chosen active ingredient.

The recommended dosage of the nucleic acid will depend on the clinical status of the subject and the experience of the physician or veterinarian in treating the viral infection or tumor burden. Specifically-configured dsRNA may be dosed at about 200 mg to about 400 mg by intravenous infusion to a 70 kg subject on a schedule of twice weekly, albeit the dose amount and/or frequency may be varied by the physician or veterinarian in response to the subject's condition. Cells or tissues that express TLR3 are preferred sites for delivering the nucleic acid, especially antigen presenting cells (e.g., dendritic cells and macrophages) and endothelium (e.g., respiratory and gastric systems). The effects of specifically-configured dsRNA may be inhibited or blocked by mutation of the TLR3 gene (e.g., deletion), down regulating its expression (e.g., siRNA), binding with a competitor for TLR3's ligand-binding site (e.g., neutralizing antibody) or a receptor antagonist, or interfering with a downstream component of the TLR3 signaling pathway (e.g., MyD88 or TRIF).

AMPLIGEN® poly(I:C₁₂U) provides a selective agent for dissecting out the effects of TLR3 activation on the immune system that was not previously available. Other agents like TLR adapters MyD88 and TRIF mediate signaling by all TLR or TLR3/TLR4, respectively. Thus, activation or inhibition of signaling through MyD88 or TRIF would not restrict the biological effects to those mediated by TLR3. Since the presence of TLR3 and its signaling is a requirement for AMPLIGEN® poly(I:C₁₂U) to act as a receptor agonist, one could assay for the absence of TLR3 mutations, the presence of TLR3 protein, intact TLR3-mediated signaling, or any combination thereof in the cell or tissue of a subject prior to administration of the agonist. Such confirmation of TLR3 activity can be performed before, during, or after administration of the agonist. The agonist can be used to restrict the immune response to activation of TLR3 without activating other Toll-like receptors or RNA helicases.

The following examples further illustrate specific embodiments of the invention and are not intended to limit its scope, which is described above.

### EXAMPLES

Punta Toro virus (PTV) is closely related by phylogeny to the viruses causing Rift Valley fever and Sandfly fever. Unlike with highly pathogenic phleboviruses, human infection with PTV produces disease that is usually limited to a mild febrile illness. Infection models in small rodents have been described which produce acute disease with hepatic involvement similar to that observed in Rift Valley Fever virus infection of humans and domesticated ungulates. Several groups have described the susceptibility of hamsters to severe disease induced by PTV infection. The availability of these rodent models makes PTV a viable alternative to the use of Rift Valley Fever virus for antiviral studies since the latter is highly restricted and requires high-level containment facilities. To that end, numerous evaluations of promising antivirals have been conducted using the PTV models of acute phlebovirus-induced disease. Moreover, several large studies have involved the evaluation of immune modulators and have demonstrated that the PTV is acutely sensitive to IFN inducers. The importance of type I IFN is borne out in the mouse PTV infection model. Treatment with neutralizing antibodies to IFN-α/β completely abolishes resistance to infection reported in adult mice. Potent type I IFN-inducers such as poly(I:C) or poly(I:C₁₂U) have consistently proven to be highly effective in protecting weanling mice from lethal PTV challenge.

There is accumulating evidence that two pathways are involved in activation events resulting from exposure to dsRNA, a replication intermediate of RNA viruses. In addition to the TLR3 response pathway, a TLR3-independent pathway mediated by RNA helicase cytoplasmic sensors that contain caspase-recruiting domains (CARDs) has recently been uncovered. Signaling by these dsRNA sensors occurs through distinct pathways that converge to share various kinases and transcriptional factors that regulate the production of IFN-β, a critical factor in regulating antiviral immunity. Due to its endosomal restriction, TLR3 is likely involved in the recognition of dsRNA that is internalized via the cellular phagocytic process. The cytosolic location of the RNA helicase dsRNA detectors, retinoic acid-induced protein I (RIG-I) and melanoma differentiation-associated gene-5 (mda-5), can sense viral infection within the cell. Recent evidence suggests that mda-5 plays a dominant role over TLR3 in the type I IFN response to poly(I:C). Here data demonstrating the role of TLR3 in the induction of protective immunity by poly(I:C₁₂U) is provided.

### Animal Subjects

TLR3^{-/-} mice were derived and backcrossed onto a C57BL/6 background at Yale University (Alexopoulou et at, Nature 413:732-738, 2001). They were bred and housed under specific pathogen-free conditions at Utah State University. C57BL/6 mice (wild type) were obtained from The Jackson Laboratory (Bar Harbor, Me.). Age-matched female mice were used in all experiments. All animal procedures used in these studies complied with guidelines set by the U.S. Department of Agriculture and the Utah State University Animal Care and Use Committee.

### Immune Modulators

Poly(I:C₁₂U) was provided by HEMISPHERx® Biopharma (Philadelphia, Pa.) at a concentration of 2.4 mg/mL. It was diluted to the appropriate concentration with sterile saline immediately prior to injection. Materials to generate cationic liposome-DNA complexes (CLDC) were provided by Juvaris BioTherapeutics, Inc. (Pleasanton, Calif.). Liposomes, DNA, and the preparation of CLDC for injection were described previously by Gowen et al. (Antiviral Res. 69:165-172, 2006). Recombinant Eimeria protozoan antigen (rEA) was provided by Barros Research Institute (Holt, Mich.) and was used as described by Gowen et al. (Anti-microbiol. Agents Chemother. 50:2023-2029, 2006). Ribavirin was supplied by ICN Pharmaceuticals (Costa Mesa, Calif.). All materials were administered by the intraperitoneal (i.p.) route.

Evaluation of Poly(I:C₁₂U) in TLR3^{-/-} and Wild-Type Mice Infected with PTV

PTV, Adames strain, was obtained from Dr. Dominique Pifat of the U.S. Army Medical Research Institute for Infectious Diseases (Frederick, Md.). Virus stocks were prepared following four passages of the original virus stock through LLC-MK₂ cells (American Type Culture Collection, Manassas, Va.), Weanling TLR3^{-/-} and C57BL/6 mice (3-4 weeks old) were inoculated by subcutaneous (s.c.) injection with 2×10⁴ 50% cell culture infectious doses (CCID₅₀) of PTV.

In the first study, a single dose of 10 μg poly(I:C₁₂U), 1 μg of CLDC, or saline placebo was administered i.p. 24 hours after infectious challenge. A ribavirin treatment group was also included in which treatment was administered twice per day for 5 days beginning 4 hours prior to viral challenge. The mice in each group were observed for death out to 21 days. In the second study, a 1 μg rEA-treatment group replaced the CLDC and ribavirin groups. Also, additional animals were included for analysis of liver disease on day 3 of infection. Serum was collected from mice (n=5) sacrificed on day 3 of the infection and livers were scored on a scale of 0 to 4 for hepatic jaundice: 0 being normal and 4 being maximal yellow discoloration. Serum alanine aminotransferase (ALT) activity was determined using the ALT (SGPT) Reagent Set purchased from Pointe Scientific, Inc. (Lincoln Park, Mich.).

A temporal study was conducted to compare systemic and liver virus loads, hepatic discoloration, and ALT levels in TLR3^{-/-} and wild-type mice treated with poly(I:C₁₂U). Groups of 8 week-old mice (n=5) were sacrificed for sample collection on day 2, 3, 4 or 5 of infection following therapeutic intervention with poly(I:C₁₂U) or saline. Day 1 samples were also collected to provide a baseline early during the course of infection. Older mice were used in this study since they are reportedly more resistant to PTV infection facilitating analysis at later stages of infection. Virus titers were assayed using an infectious cell culture assay as described by Sidwell et al. (Antimicrob. Agents. Chemother. 32:331-336, 1988). Briefly, a specific volume of liver homogenate or serum was serially diluted and added to triplicate wells of LLC-MK₂ cell monolayers in 96-well microplates. Viral cytopathic effect (CPE) was determined six days post-virus exposure and the 50% endpoints were calculated as described by Reed & Muench (Am. J. Hyg. 27:493-497, 1938).

### IFN-β in TLR3^{-/-} and Wild-Type Mice Following Poly(I:C₁₂U) Treatment

Groups of 8 week-old TLR3^{-/-} and wild-type mice (n=3 per group) were treated with 10 μg poly(I:C₁₂U). Serum was collected at 0, 1.5, 3, 6 and 24 hours post-exposure. Systemic IFN-β levels were measured using ELISA reagents from PBL (Piscataway, N.J.) as specified by the manufacturer.

### Statistical Analysis

Log-rank analysis was used to evaluate differences in survival data. The Fisher's exact test (two-tailed) was used for evaluating increases in total survivors. The Mann-Whitney test (two-tailed) was performed to analyze the differences in mean day to death, virus titers, and serum ALT levels. Wilcoxon ranked sum analysis was used for mean liver score comparisons. The Student's t-test (two-tailed) was used to determine differences in IFN-β levels between TLR3^{-/-} and wild-type mice treated with poly(I:C₁₂U).

TLR3-Deficient Mice Fail to Develop Protective Immunity to PTV Infection Following Treatment with Poly(I:C₁₂U)

Poly(I:C₁₂U) is a drug that has previously been reported to confer complete protection in C57BL/6 weanlings against lethal PTV challenge, reduce virus titers, and limit liver dysfunction and disease associated with PTV infection (Sidwell et al., Ann. N.Y. Acad. Sci. 653:344-35, 1992). To determine whether TLR3 activity plays an important role in the induction of antiviral defenses against PTV by poly(I:C₁₂U), 3-4 week-old TLR3^{-/-} and wild-type mice were treated 24 hours post-infectious challenge in a first study. There were no survivors in the TLR3^{-/-} group of mice treated with poly(I:C₁₂U) (Table 1). In contrast, five of eight mice stimulated with CLDC, which likely acts primarily via TLR9 recognition of CpG motifs present in the plasmid DNA backbone, survived the infection. In the wild-type mice, both the dsRNA and CLDC protected 100% of the mice (Table 1), verifying that the immunomodulatory drug preparations were highly active. Ribavirin treatmeat was also included as an additional positive control since it routinely protects 90% or more of wild-type mice from lethal PTV challenge, Notably ribavirin only protected 75% (six of eight mice) of the TLR3^{-/-} mice from death whereas complete protection was observed in wild-type animals (Table 1). This may have been due to the smaller TLR3^{-/-} mice (˜3 weeks of age) used compared to the slightly larger wild-type mice (˜3-4 weeks of age). Alternatively, the TLR3 deletion may reduce the capacity of these mice to limit the infection and combat the disease. Both CLDC and ribavirin significantly improved survival outcome.

TABLE 1
CLDC, but not mismatched dsRNA, poly(l:C₁₂U) elicited protective immunity
to PTV infection in mice devoid of TLR3.

| | | | Day of Death^{b} | | Log-Rank |
|---|---|---|---|---|---|
| Strain | Treatment^{a} | Alive/Total | Mean ± SD | Range | Prob > Chi Sq |
| TLR3^{-/-} | poly(l:C₁₂U), | 0/9 | 4.1 ± 0.3 | 4-5 | 0.6775 |
| | 10 μg | | | | |
| | CLDC, 1 μg | 5/8** | 3.7 ± 0.6 | 3-4 | 0.0163 |
| | Ribavirin, 75 mg/kg/ | 6/8** | 6.0 ± 2.8 | 4-8 | 0.0003 |
| | day | | | | |
| | Sterile saline | 0/9 | 4.2 ± 1.0 | 3-6 | |
| Wild-type | poly(l:C₁₂U), | 10/10*** | | | <0.0001 |
| | 10 μg | | | | |
| | CLDC, 1 μg | 10/10*** | | | <0.0001 |
| | Ribavirin, 75 mg/kg/ | 10/10*** | | | <0.0001 |
| | day | | | | |
| | Sterile saline | 1/11 | 4.5 ± 0.7 | 4-6 | |

^{a}Single-dose poly(l:C₁₂U), CLDC, and saline treatments administered i.p. 24 hours post-virus challenge. Ribavirin given i.p. twice per day for five days beginning four hours pre-virus challenge.
^{b}Mean and range day of death of mice dying prior to day 21.
*P < 0.05;
**P < 0.01;
***P < 0.001 compared to the respective saline-treated controls.

A second study was conducted to verify the initial findings. In addition to having more closely age-matched mice (˜4 weeks of age), five additional mice were included for sacrifice on day 3 of infection in order to assess differences in liver disease as a consequence of PTV infection. As shown in Table 2, poly(I:C₁₂U) failed again to protect TLR3^{-/-} mice from a highly lethal dose of virus and failed to limit liver disease as reflected by reduced levels of serum ALT and liver scores, rEA, a positive control immune modulator that acts through TLR11, was highly effective at protecting mice from death and significantly reducing serum ALT levels. As expected, treatment of wild-type mice with poly(I:C₁₂U) and rEA elicited 100% protection against the highly lethal challenge inoculum (Table 2). Interestingly poly(I:C₁₂U) therapy, which is known to induce type I IFN, dramatically abrogated hepatic jaundice while rEA, which has not been shown to induce type I IFN, did not reduce mean liver scores in either strain of mouse. There were no significant differences when comparing the TLR3^{-/-} and wild-type saline-treated placebo and rEA treatment groups suggesting that both strains were equally susceptible to PTV infection and responded similarly to rEA.

TABLE 2
TLR11 agonist, rEA, but not mismatched dsRNA, poly(l:C₁₂U),
protected TLR3-deficient mice from lethal PTV disease.

| | | | Day of Death^{b} | | Log-Rank | | |
|---|---|---|---|---|---|---|---|
| | | | Mean ± | | Prob > Chi | | Liver |
| Strain | Treatment^{a} | Alive/Total | SD | Range | Sq | ALT^{c,d} ± SD | Score^{c,e} ± SD |
| TLR3^{-/-} | poly(l:C₁₂U) | 0/10 | 4.1 ± 0.6 | 3-5 | 0.4861 | 2700 ± 1576 | 3.2 ± 0.4 |
| | 10 μg | | | | | | |
| | rEA, 1 μg | 10/10*** | | | <0.0001 | 155 ± 77** | 3.3 ± 0.3 |
| | Sterile | 1/10 | 4.1 ± 0.6 | 3-5 | | 3837 ± 234 | 3.5 ± 0.0 |
| | saline | | | | | | |
| Wild- | poly(l:C₁₂U) | 10/10*** | | | <0.0001 | 3 ± 6** | 0.6 ± 0.2** |
| type | 10 μg | | | | | | |
| | rEA, 1 μg | 10/10*** | | | <0.0001 | 93 ± 56** | 3.3 ± 0.3 |
| | Sterile | 1/20 | 4.8 ± 1.1 | 3-7 | | 3650 ± 823 | 3.2 ± 0.3 |
| | saline | | | | | | |

^{a}Singe-dose poly(l:C₁₂U), rEA, and saline treatments administered i.p. 24 h post-virus challenge.
^{b}Mean and range day of death of mice dying prior to day 21.
^{c}Determined on day 3 of infection; 4-5 mice per group.
^{d}ALT, alanine aminotransferase; measured in international units per liter.
^{e}Score of 0 (normal liver) to 4 (maximal discoloration).
*P < 0.05;
**P < 0.01;
***P < 0.001 compared to respective saline-treated controls.

### TLR3-Deficient Mice Fail to Reduce Disease Severity and Viral Load in Response to Poly(I:C₁₂U)

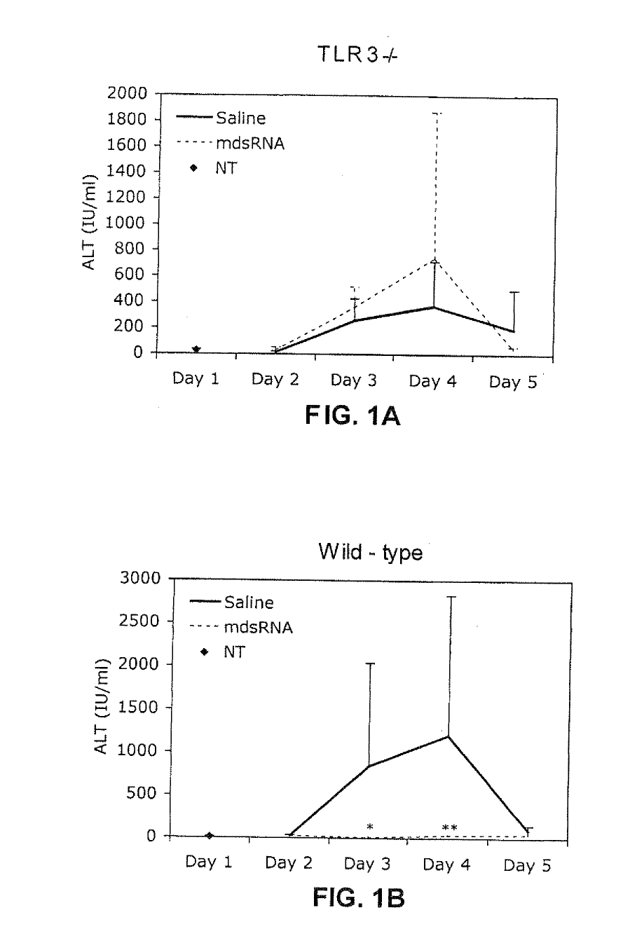

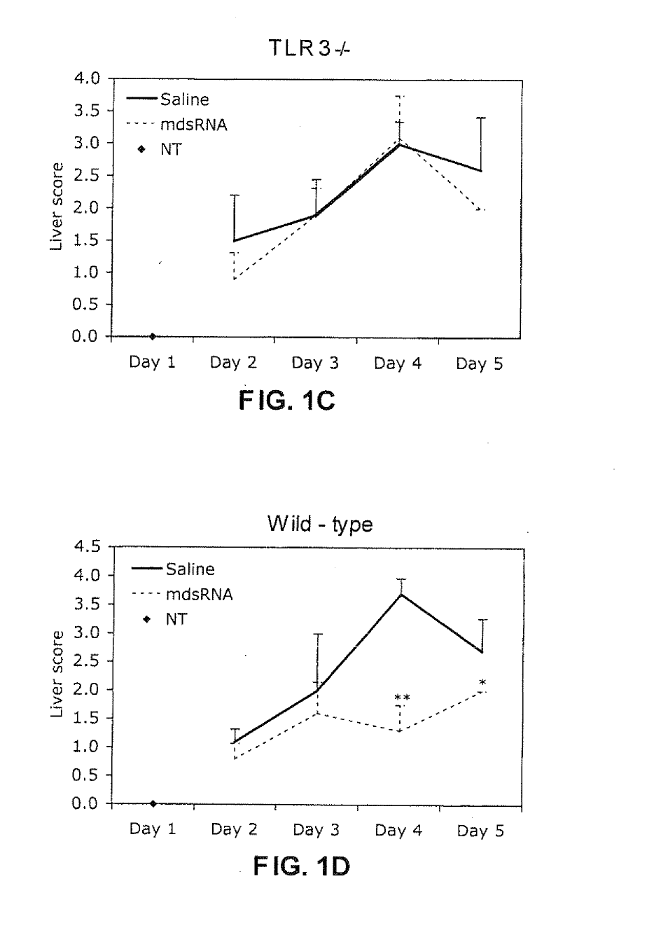

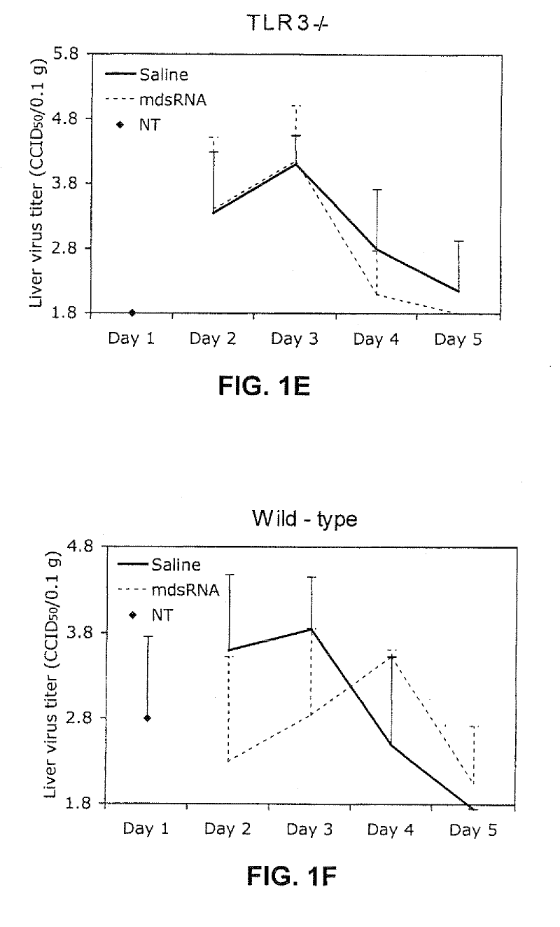

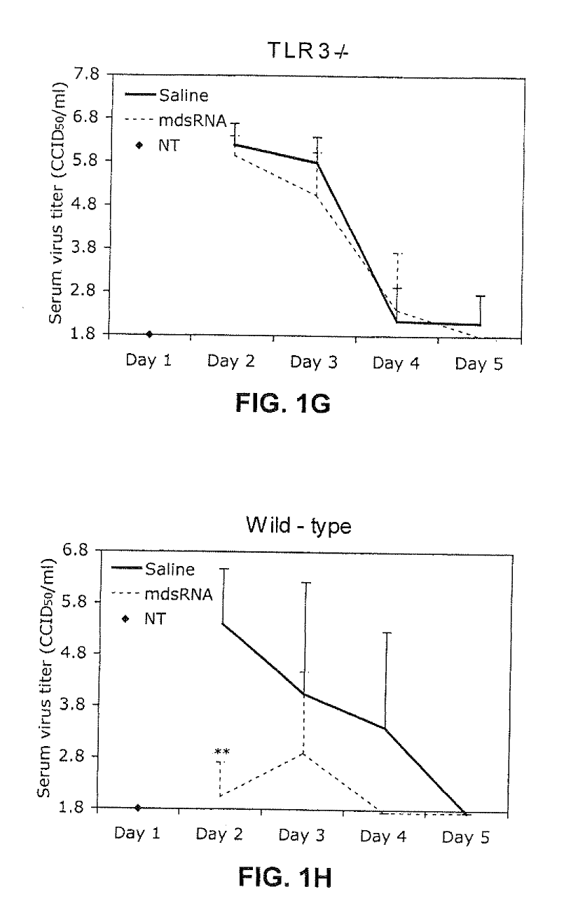

Although PTV is highly lethal in weanling C57BL/6 mice, 8 week-old animals are reportedly refractory to infection. Thus, a time course study was conducted spanning the entire infection and disease process using older mice in order to further evaluate the contribution of TLR3 to the protective effect of poly(I:C₁₂U) immunotherapy. As seen in FIG. 1A, remarkable levels of ALT were not present until day 3 of infection in the TLR3^{-/-} and wild-type mice and peaked on day 4 before going back down. There were no differences in ALT levels between poly(I:C₁₂U)-treated and saline-treated TLR3^{-/-} mice whereas levels remained near baseline in the wild-type mice that received dsRNA therapy (FIGS. 1A-1B). Interestingly, despite the large variation seen on days 3 and 4 of infection, saline-treated wild-type mice presented with mean ALT levels three times greater than their TLR3-deficient counterparts. For liver damage assessed by gross visual examination, disease indicated by discoloration was first noted on day 2 and peaked on day 4 in saline-treated mice (FIGS. 1C-1D). In concordance with the liver disease reflected by the ALT data, a significant reduction in hepatic jaundice compared to the saline treatment control on days 4 and 5 was only demonstrated in wild-type mice responsive to poly(I:C₁₂U). Again, the suggestion of greater liver disease severity in the wild-type mice was observed as they had higher day 4 mean liver scores compared to the TLR3^{-/-} mice (3.7±0.3 and 3.4±0.4, respectively). And consistent with the lack of protection seen in the previous challenge studies (Tables 1 and 2), the data indicate that TLR3 plays a vital role in mediating protective immunity against PTV following poly(I:C₁₂U) treatment.

Control of liver and systemic viral burden during the course of infection following poly(I:C₁₂U) or saline treatment was also examined. Unexpectedly, no appreciable differences in liver viral loads were found, in part due to the high degree of variability seen with the wild-type mice (FIGS. 1E-1F). The mean titers were higher on days 2 and 3 in the poly(I:C₁₂U)-treated wild-type mice, but not statistically significant as demonstrated in several cases with serum ALT levels and liver scores. Notably, in contrast to the TLR3^{-/-} mice, virus was detected as early as day 1 in several wild-type animals (FIGS. 1E-1F). Although not detectable on day 1 of the infection, serum virus spiked dramatically by day 2, with the exception of the dsRNA-treated wild-type mice which were able to control the infection to barely detectable levels as shown by a greater than three-log reduction of virus (FIGS. 1G-1H), Once again, the effect of poly(I:C₁₂U) therapy seen in the wild-type animals was lost in the TLR-deficient mice. As with the comparison of liver viral loads in TLR3^{-/-} and wild-type mice, no significant differences were seen systemically in peak titers for the saline-treated groups (FIGS. 1G-1H). But mean virus titers in TLR3^{-/-} mice dropped precipitously by more than three-logs after day 3, while only a gradual decrease was observed in the wild-type. The comparison between the saline-treated groups of mice corroborates the suggested more severe liver disease profile seen with the wild-type animals. Liver virus was detectable earlier (day 1) and systemic virus persisted longer in the wild-type animals (FIGS. 1E-1H).

### TLR3-Deficient Mice do not Produce IFN-β in Response to Poly(I:C₁₂U) Treatment

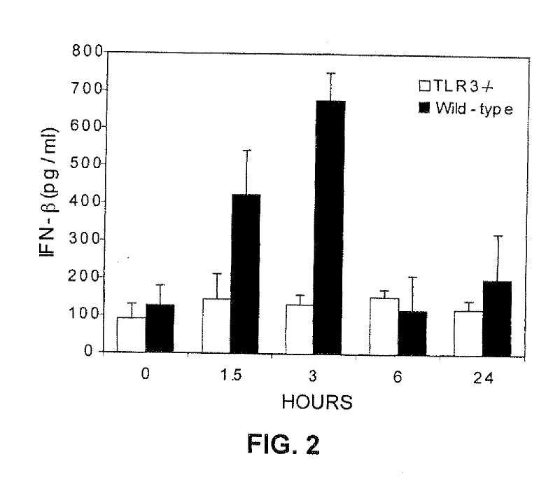

dsRNA is a major inducer of IFN-β, a critical factor in the establishment of the host antiviral defenses. To test whether lack of functional TLR3 alters the IFN-β response profile, groups of wild-type and TLR3^{-/-} mice were treated with the 10 μg poly(I:C₁₂U) dosage used in all experiments and systemic IFN-β production was determined at various time points. Following a 1.5 hour exposure period, a significant increase in IFN-β levels was observed in wild-type mice compared to the TLR3^{-/-} mice (FIG. 2). At 3 hours, IFN-β levels peaked in the wild-type mice while remaining at basal levels in the TLR3^{-/-} mice. By 6 hours, IFN-β levels had returned to baseline in the wild-type mice (FIG. 2). There was no indication of IFN-β induction at any of the time points evaluated for the TLR3^{-/-} mice. The differences in IFN-β production at the 1.5 and 3 hour sampling times were significant and likely factor in the inability of poly(I:C₁₂U) to elicit protective immunity against PTV infection in TLR3-deficient animals.

### Discussion

There are several lines of evidence that argue against dsRNA-dependent protein kinase (PKR), the classic cytosolic sensor for dsRNA, being the prominent pathway for type I IFN induction and antiviral host defense. The above results show that pattern recognition receptor TLR3 is essential for protective immunity elicited by poly(I:C₁₂U) in mice. The recent discoveries of other cytoplasmic dsRNA sensors and their involvement in host antiviral immunity suggest that mda-5 is the predominant mechanism for type I IFN induction, and the resulting antiviral state. It was found, however, that animals devoid of TLR3 failed to develop protective immunity against, and limit disease associated with, PTV infection following a single-dose treatment with poly(I:C₁₂U), Moreover, TLR3 deficiency resulted in unchecked viral replication and the absence of an IFN-β response clearly evident in wild-type animals treated with poly(I:C₁₂U).

A caveat associated with antiviral studies in mice with immunodeficiencies such as TLR3 deletion is that lack of efficacy may be due in part to disruption of a TLR3-mediated response to PTV infection independent of poly(I:C₁₂U). To that end, it is conceivable that TLR3 depletion predisposes the mice to more severe disease and consequently a more difficult to treat infection. The results from the first study (Table 1) suggested that this may be the case since the positive control drugs ribavirin and CLDC, which normally protect 100% and greater than 80% of challenged mice, respectively, were less effective. These results may, however, have been influenced by the age of the TLR3^{-/-} mice, which were noticeably smaller and presumably a few days younger than the wild-type mice in this experiment. This explanation is supported by the results from the second study, where the mice were more rigorously age-matched so that they would all be close to 4 weeks of age. Indeed, very similar protection was seen among the two mice strains in response to rEA and similar lethality was observed with the saline placebo groups (Table 2). Thus, further evidence refuting the diminished capacity of TLR3^{-/-} mice to combat PTV infection was also seen in older mice. In the time course study that was conducted to further resolve differences in the ability of TLR3^{-/-} and wild-type mice to respond to poly(I:C₁₂U), comparison between the placebo-treated mice suggest that the TLR3^{-/-} mice may be more resistant to PTV infection and disease. Wild-type mice presented with higher levels of ALT and liver scores and, despite having similar peak serum titers, virus persisted longer showing a gradual reduction whereas titers in TLR3^{-/-} mice dropped abruptly after day 3 of infection. Additional challenge studies in untreated TLR3^{-/-} and wild-type mice corroborate these findings indicating that TLR3^{-/-} mice are no more susceptible to PTV infection than their wild-type counterparts.

A recent investigation into the mechanism underlying the host response to the commonly used dsRNA mimic, poly(I:C), provides compelling evidence that the cytosolic dsRNA sensor, mda-5, is the primary response pathway to type I IFN production. The above results suggest that TLR3 is the dominant dsRNA response mechanism. It is essential for antiviral activity and induction of IFN-β. Several issues come to mind when comparing these findings with those of Gitlin and colleagues. In their studies, 100 μg poly(I:C) was administered by intravenous i.v. injection while treatment in this study was limited to 10 μg of poly(I:C₁₂U) administered by the i.p. route. The 10 μg amount was based on experiments designed to determine the most appropriate dose for maximal antiviral activity in the PTV infection model. Presumably, the composition of the dsRNA, its route of administration, and the amount inoculated contributed significantly to the discrepancies observed in the type I IFN responses. It is plausible that mda-5 has greater specificity for the poly(I:C) form of dsRNA while TLR3 has greater affinity for poly(I:C₁₂U). Also the route of delivery is important in that there appears to be cell-type specific differences in the recognition of dsRNA. By delivering dsRNA i.v. the material is initially accessed in the marginal zone of the spleen populated by dendritic cells (DCs) that do not express significant levels of TLR3, thereby resulting in predominantly mda-5 mediated type I IFN induction. In contrast, i.p. administration results in initial encounter by resident and infiltrating inflammatory peritoneal macrophage populations where TLR3-mediated activation appears to be the major pathway used. This idea is supported by a number of ex vivo studies that have explored dsRNA responses by TLR3- and TRIF-deficient peritoneal macrophages in culture. Thus, direct exposure of macrophages to dsRNA in the peritoneal cavity produces a different response profile than what would be observed when exposure occurs through direct systemic delivery targeting CD8-negative DCs suited to mda-5 mediated type I IFN induction.

In addition to the above potential sources contributing to the observed differences between this study and Gitlin and coworkers, the amount of dsRNA used was significantly different. Here, 10-fold less dsRNA was used, which combined with the i.p. route of delivery, resulted in comparably lower levels of systemic IFN-β. Administration of 100 μg of naked dsRNA by the i.v. route to produced more than 10-fold excess IFN-β than what was observed in this study (cf. Gitlin at al., Proc. Natl. Acad. Sci. USA 103:8459-8464, 2006). Nevertheless, the amount of IFN-β induced by the lower dose was more than sufficient to induce protective immunity in the PTV infection model. In fact, one μg or less of poly(I:C₁₂U) material still provides adequate protection against lethal PTV challenge, which is more likely physiologically relevant in the context of viral infection and potential immunotherapy with poly(I:C₁₂U). The latter is especially important considering the known toxicity of poly(I:C). Presumably, the inability to produce type IFN-β after exposure to i.p.-administered poly(I:C₁₂U) was vitally detrimental to the outcome of the challenge studies with the TLR3^{-/-} mice.

Patents, patent applications, books, and other publications cited herein are incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim which recites “comprising” allows the inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification to should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrange-ment of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of initiating an innate immune response mediated only by Toll-Like Receptor 3 (TLR3), said method comprising administration to a subject of at least poly(I:C₁₂U) in an amount sufficient to activate TLR3 without activating other Toll-like receptors or RNA helicases.

2. A method of treating a subject (i) infected with a virus or (ii) bearing a tumor or other transformed cell, said method comprising administration of a pharmaceutical composition comprised of poly(I:C₁₂U) in an amount sufficient to bind to Toll-Like Receptor 3 (TLR3) to reduce or eliminate (i) infection of the subject by the virus or (ii) proliferation of the tumor or other transformed cell in the subject, respectively.

3. The method according to claim 2, wherein the subject is infected with a virus.

4. The method according to claim 3, wherein the virus is a bunyavirus.

5. The method according to claim 4, wherein the virus is a phlebovirus.

6. A method of vaccinating a subject against a virus or a tumor, said method comprising administration of (i) a vaccine which induces an immune response against the virus or the tumor and (ii) a pharmaceutical composition comprised of poly(I:C₁₂U) in an amount sufficient to bind to Toll-Like Receptor 3 (TLR3) and to stimulate the immune response against a viral or tumor antigen of the vaccine in the subject.

7. The method according to claim 6, wherein the subject is vaccinated against a virus.

8. The method according to claim 7, wherein the virus is a bunyavirus.

9. The method according to claim 8, wherein the virus is a phlebovirus.

10. The method according to claim 1, wherein the subject is a human.

11. The method according to claim 2, wherein the virus or the tumor is susceptible to the sole action of poly(I:C₁₂U) acting exclusively as a TLR3 agonist.

12. The method according to claim 2, wherein the virus or the tumor expresses an antigen that is spontaneously selected by poly(I:C₁₂U) as an in situ target to initiate an immune response against the antigen.

13. The method according to claim 1, wherein poly(I:C₁₂U) is infused intravenously.

14. The method according to claim 1, wherein poly(I:C₁₂U) is injected intradermally, subcutaneously, or intramuscularly; inhaled intranasally or intratracheally; or exposed oropharyngeally.

15-16. (canceled)

17. The method according to claim 6, wherein the virus or the tumor is susceptible to the sole action of poly(I:C₁₂U) acting exclusively as a TLR3 agonist.

18. The method according to claim 6, wherein the virus or the tumor expresses an antigen that is spontaneously selected by poly(I:C₁₂U) as an in situ target to initiate an immune response against the antigen.

19. The method according to claim 2, wherein poly(I:C₁₂U) is infused intravenously.

20. The method according to claim 6, wherein poly(I:C₁₂U) is infused intravenously.

21. The method according to claim 2, wherein poly(I:C₁₂U) is injected intradermally, subcutaneously, or intramuscularly; inhaled intranasally or intratracheally; or exposed oropharyngeally.

22. The method according to claim 6, wherein poly(I:C₁₂U) is injected intradermally, subcutaneously, or intramuscularly; inhaled intranasally or intratracheally; or exposed oropharyngeally.
